(19) United States
(12) Patent Application Publication
(10) Pub. No.: US 2023/0173289 A1
(43) Pub. Date: Jun. 8, 2023

(54) PULSED EMF LUMBAR DECOMPRESSION DEVICE

(71) Applicant: Excite Medical of Tampa Bay, LLC, Tampa, FL (US)

(72) Inventors: Scot Leslie Johnson, Tampa, FL (US); Saleem Najeeb Musallam, Tampa, FL (US)

(21) Appl. No.: 17/983,244

(22) Filed: Nov. 8, 2022

Related U.S. Application Data
(60) Provisional application No. 63/277,151, filed on Nov. 8, 2021.

(51) Int. Cl. A61N 2/00; A61N 2/02; A61H 1/02

(52) U.S. Cl. CPC A61N 2/002; A61N 2/02; A61H 1/0229; A61H 2201/0146; A61H 2203/045; A61H 2001/0233; A61H 2201/1628; A61H 2201/1652; A61H 2201/10; A61H 2205/081

(57) ABSTRACT

A spinal therapy device combining pulsed electromagnetic frequency (PEMF) technology and lumbar spinal decompression therapy. The combined device can provide consistent PEMF therapy to a patient who is also undergoing simultaneous lumbar spinal decompression therapy. The device configuration utilizes lumbar spinal decompression methods, which are advantageous for application of PEMF to the lumbar spine.

### FIELD OF THE PRESENT DISCLOSURE

The present disclosure generally relates to the field of spinal decompression therapy and in particular to a device for applying pulsed energy to the spine during spinal decompression therapy.

### BACKGROUND OF THE PRESENT DISCLOSURE

The mature human spine is made of 24 separate vertebral bones (5 lumbar, 12 thoracic, 7 cervical), separated by 23 cartilaginous intervertebral discs. Each intervertebral disc is a major load-bearing joint. Additionally, there are over 100 smaller cartilage-bearing facet joints arranged about the vertebral bones which provide structure and support for smooth spinal movements. The synovium and its associated products, e.g., synovial fluid, provide lubrication and cushioning for the joints of the spine. Over 100 muscles and 220 individual ligaments work with the spine during normal function.

As the health of the spine degrades, patients may initially experience back pain associated with movement. The pain generally follows execution of movement which had not previously caused back pain, e.g., lifting a child. This is sometimes broadly characterized as osteoarthritis (OA). Chronic injury to the spine results in degradation of cartilage, reduction of joint space, narrowing of passageways through which nerves transit, inflammation, pain, and loss of function. Eventually, little-to-no fluid or material, e.g., cartilage, separates the opposing faces of bone which grind upon each other during movement. In the final stages of chronic injury, the bones of the joint naturally fuse together, a process familiar to any reader who has experienced a broken bone.

Prior to natural bone fusion or total loss of joint function, the spine naturally attempts to restore homeostasis with the external environment through spinal remodeling. The human spine naturally maintains a homeostatic balance with the external environment through constant remodeling of its cartilage, bone and synovium either anabolically or catabolically. Anabolic remodeling is initiated in response to movement and increased load-bearing demands, synthesizing healthy new tissues and suppressing inflammatory processes. Catabolic remodeling is initiated in the absence of mechanical movement or stress, promoting inflammatory processes and wasting unnecessary tissue for reintegration or elimination from the body.

Anabolic and catabolic remodeling of the human body is a process all readers are familiar with. When an individual regularly performs healthy exercise, their body undergoes anabolic changes which include strengthening muscles, bones and joints. The increase in density and strength of muscle, bone and connective tissue is the result of anabolic growth, synthesis of new muscle/bone/connective tissue. When an individual rarely performs any exercise, their body undergoes catabolic changes which include wasting of muscle strength and size along with weakening of bones and joints. The decreases in the size and density of these tissues are the result of catabolic destruction of these tissues and the resorption into the body of their constituent components.

The ability of the spine to repair itself anabolically declines with age, in the absence of routine use, and due to injury. Routine load-bearing movements and exercise of the range-of-motion of the spine's intervertebral and facet joints, within healthy limits, encourages an anabolic environment and reduces the impacts of aging. Conversely, whether by age, disuse or injury, joint and bone tissues subjected to chronic catabolic and inflammatory exposure become incapable of supporting sufficient clearance for nerves to pass through the spine unimpinged, resulting in pain, loss-of-function and further inflammatory signaling. Additionally, they can become incapable of movement within their intended range-of-motion. Finally, they can become incapable of separating the faces of the bones of the joint from direct contact, resulting in gross damage and pain, and ultimately natural fusion of the bones.

Despite modern surgical and pharmacological interventions, debilitating low back pain (LBP) is reported at increasing rates per capita year-over-year, and not less. Whether surgical and pharmacological interventions can ever fully eliminate lower back pain is yet to be demonstrated.

### SUMMARY OF THE DISCLOSURE

According to one embodiment of the present disclosure, the disclosure relates to a pulsed electromagnetic field spinal decompression device. The device includes a generally-horizontal mattress having a first end and a second end. A generally-vertical decompression tower is disposed at the first end of the generally-horizontal mattress. A tension strap having a first end and second end, is operably connected to the decompression tower at its first end. A pelvic harness is operably connected to the second end of the tension strap. A pulsed electromagnetic field device is disposed in the generally-horizontal mattress.

The foregoing summary, as well as the following detailed description of certain embodiments of the present disclosure, will be better understood when read in conjunction with the appended drawings. Various objects, features, aspects and advantages of the inventive subject matter will become more apparent from the following detailed description of disclosed embodiments, along with accompanying drawing figures in which like numerals represent like components.

### BRIEF DESCRIPTION OF THE DRAWING FIGURES

The foregoing disclosure will be best understood, and the advantages thereof made most clearly apparent, when consideration is given to the following detailed description in combination with the drawing figures presented. The detailed description makes reference to the following drawings:

FIG. 1 is a side view of a spinal decompression/PEMF therapy device according to the present disclosure;

FIG. 2 is a close-up view of the Apex-of-Lordosis (AoL) 108 region of a patient's spine positioned above the proximal-end of the upper mattress section 110 of the split-mattress 106 shown in FIG. 1;

FIGS. 3A and 3B are oblique and top views, respectively, of one embodiment of a “twin-coil” 300 used to produce pulsed electromagnetic fields;

FIG. 4 is a side view of twin-coil 300 shown in FIGS. 3A and 3B lying flat, with wire ends 314 and 316 extending towards the viewer;

FIGS. 5A, 5B, 5C and 5D are oblique, top, end and right side views, respectively, of one embodiment of PEMF device 152, positioned such that axes 304 and 312 of twin-coil 300 are essentially normal to the surface of the upper-mattress section 110 of the split-mattress 106;

FIGS. 6A and 6B are front-left side views of one embodiment of PEMF device 152 mounted upon a section 602 of upper frame 154;

FIG. 7 is an exploded view of one embodiment of the present disclosure;

FIGS. 8A and 8B are front views of one embodiment of the present disclosure;

FIGS. 9A and 9B are front views of one embodiment of the present disclosure;

FIGS. 10A and 10B are left-side section views of PEMF device 152 cut down the center lengthwise plane of PEMF module 604; and

FIG. 11 is a top-down view of PEMF device 152.

For the purpose of illustrating the disclosure, certain embodiments are shown in the drawings and described herein. It should be understood, however, that the present disclosure is not strictly limited to the arrangements and instrumentalities shown in the attached drawings.

### DETAILED DESCRIPTION OF THE DISCLOSURE

The following detailed description provides certain specific embodiments of the subject matter disclosed herein. Although each embodiment represents a single combination of elements, the subject matter disclosed herein should be understood to include sub-combinations of the disclosed elements. Thus, if one embodiment comprises elements A, B, and C, and a second embodiment comprises elements B and D, then the inventive subject matter is also intended to include other remaining combinations of A, B, C, or D, even if not explicitly disclosed herein.

One embodiment of the present disclosure comprises a pulsed electromagnetic force (PEMF) device incorporated into the mattress of a supine-orientation-type spinal decompression device. The disclosure can be utilized for either PEMF or spinal decompression therapy, or both.

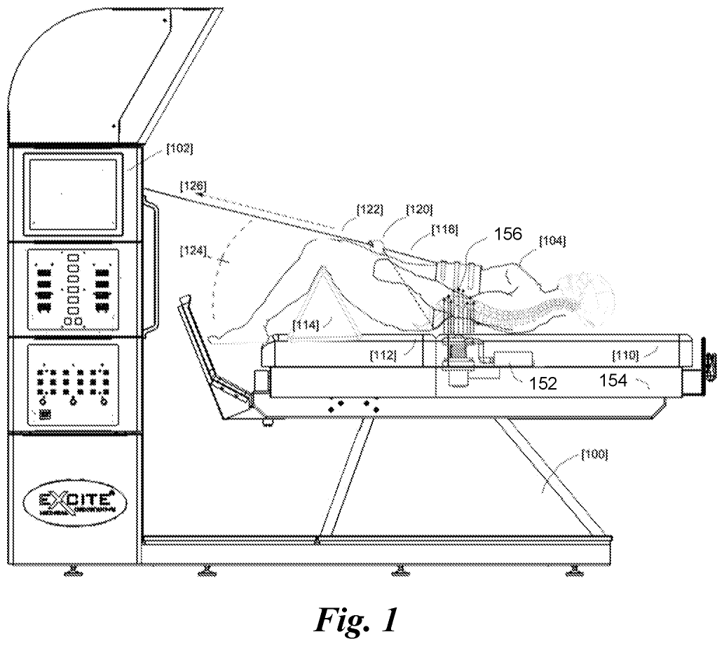

FIG. 1 depicts a spinal decompression/PEMF spinal therapy device 100 according to the present disclosure. Device 100 orients the patient in a supine position, in which the feet are proximal to the decompression tower 102. A patient 104 is shown laying supine upon a split mattress 106, also typical of modern spinal decompression technology. The mattress in this figure is split according to an anthropometric landmark, the ‘Apex of Lordosis’ (AoL) 108. Spinal decompression devices of this type place the AoL over the end of the upper mattress section 110 as shown, and portions of the sacrum and coccyx above the lower mattress section 112. A knee support 114 is often utilized as a positioning aide and for patient comfort.

A pelvic harness 116 is shown, worn about the pelvis region of the patient. The harness, typically of textile construction, conforms to the lower torso and hips of the patient and is held tight by fasteners (e.g., straps and clips). Pelvic harness 116 is shown connected by multiple straps 118 to a ring 120, and the ring to a tension strap 122 which feeds into decompression tower 102. Tension strap 122 is shown raised at an angle 124 relative to the surface of the mattress, as is typical of targeted distractive forces utilized in spinal decompression therapy. A dashed arrow 126 is shown above the tension strap indicating the direction of the applied distractive force during a treatment session.

Spinal decompression/PEMF spinal therapy device 100 incorporates PEMF device 152 for application of one or more pulsed electromagnetic fields to the therapeutic region. PEMF device 152 is shown mounted to upper frame 154 and within the upper mattress section 110 of device 100. In this embodiment, the lumbar spine apex-of-lordosis 108 is located approximately over a twin-coil within PEMF device 152, as described in detail below. While spinal decompression tower 102 is pulling on tension strap 120 generating distractive force 126, PEMF device 152 is generating pulsed electromagnetic frequencies 156 which extend into, through and surrounding the lumbar spine. In this configuration, the patient may receive both spinal decompression and PEMF therapy simultaneously or sequentially.

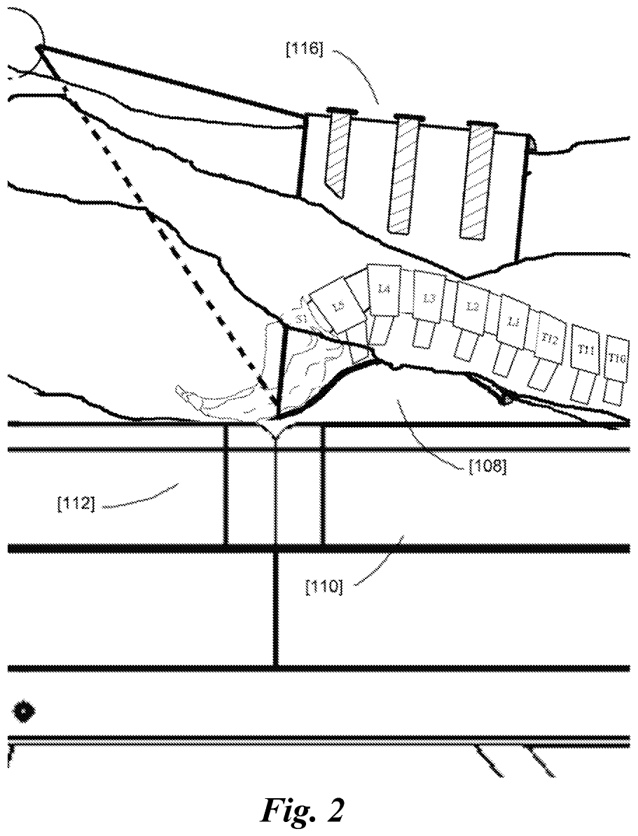

FIG. 2 is a close-up view of the AoL 108 region positioned above the proximal end (relative to the decompression tower 102 not shown) of the upper mattress section 110 of the split mattress 106. The lower mattress section 112 is shown underneath the coccyx of the patient. A pelvic harness 116 is shown cinched about the patient's 104 mid-section.

“Traction” is the activity of mechanical unloading of the spine through constant, or steady-state, application of one or more opposing forces in communication with said spine. “Decompression” is the activity of the mechanical unloading of the spine, through cyclical application of one or more opposing forces in communication with said spine and which forces preferably affect an intended sub-region of the spine.

“Cyclical” as used in reference to “decompression” means to modulate the amplitude, or intensity, of forces distracting the spine, as used herein. Mechanical unloading, in either case, is intended to distract the vertebral bones and facet joints resulting in elongation of the spine, which in turn leads to induction of a negative pressure-gradient upon and within the region.

Elongation of the spine is intended generally to reduce or eliminate pain and loss-of-function from compression of intervertebral discs, facet joints, and nerves transiting the spine. Secondarily, the resultant pressure gradient about and within the elongated region is generally intended to: (a) facilitate rehydration of the spine, as the satisfaction of the pressure gradient is the exchange of nutrient-rich fluids from tissues adjacent to the region of the spine; and (b) facilitate movement of herniated materials back into the elongated region of the spine, which also satisfies the induced pressure gradient.

Especially mobilized via the negative pressure-gradient are those fluids, e.g., herniated spinal nucleus adjacent to the damaged annulus, in most direct communication with the elongated spine. Herniated nucleus is drawn back through the damaged annulus, reducing the size of the herniation and its impingement-effect zone. Reincorporation of herniated nucleus reduces strain and inflammatory signaling in the region of damaged annulus, facilitating normal closure there.

Typical presentation of back pain includes one or more regions of inflamed and compressed intervertebral disc and facet joint spaces. Compression may be gradual or from traumatic insult. Repeated mechanical unloading of the spine is intended to generally restore normal intervertebral disc and facet joint height, relative to the compressed state of the spine prior to any traction or decompression therapy.

“Decompression” is commonly differentiated from “traction” by two characteristics. First is that it is cyclic, as opposed to steady-state, mechanical unloading of the spine. Second is the incorporation of mechanical systems intended to apply decompressive force(s) which preferably target regions of the spine of pathological interest.

Cycling the intensity of the mechanically-distractive force(s) generates oscillating negative pressure gradients upon the cells of and within the spine. The ‘pumping action’ of the cyclical mechanical unloading employed by “decompression” therapy is intended to enhance nutrient-rich rehydration of the spine. For example, enhanced rehydration of intervertebral discs, during each decompression therapy session, reduces the overall time and number of treatments required to achieve intervertebral-disc height(s) sufficient to support acceptable patient outcomes.

Cycling the intensity of the mechanical unloading additionally creates a state of ‘muscle confusion’, preventing paraspinal muscle contraction triggered by prolonged or excessive elongation.

The degree to which the spine can be mobilized influences the ease with which an anabolic environment can be induced mechanically, either by “traction” or “decompression” therapy. This brings to light a third and perhaps most distinguishing attribute of “decompression” therapy (as compared to “traction”). Cycling the intensity of the forces applied to the spine generates flexion of both the facet and intervertebral joint spaces, all while in a continuously unloaded state, triggering upregulated anabolic signaling there. The importance of this should not be lost on the reader. In a majority of cases, movement of these joints is no longer possible in the normally loaded, compressed state of the spine of the patient.

Biochemically speaking, growth and repair of both cartilage, bone and synovial tissue begins with synthesis of their respective extracellular matrix (ECM). Generally, the ECM is a three-dimensional space which contains proteoglycans and other macromolecules which are the constituents of human tissues, including cartilage, bone, and synovial products. Synthesis of new ECM requires synthesis of new proteoglycans. These are molecules that (a) fill the spaces of the ECM between other macromolecules and particles therein, (b) other macromolecules adhere to and through which integrate into the ECM and (c) generate lubrication, dampening, and absorption associated with mechanical movement, shock and stress.

Cartilage is comprised primarily of proteoglycan and collagen fibers. Both proteoglycans and collagen derive from chondroblasts which generate the ECM of cartilage. Similarly, osteoblasts are responsible for the ECM of bone which is comprised of collagen, bone minerals and proteoglycans. In this respect, proteoglycan synthesis is one direct correlation to cartilage and bone repair. The body protects the process of proteoglycan synthesis through the production and release of anti-inflammatory cytokines.

Revisiting the discussion of anabolic and catabolic activities as they relate to the remodeling of the spine, anabolic activities that occur within and to the ECM include electrochemical signaling and subsequent release of anti-inflammatory cytokines, and transformation of human mesenchymal stem cells (MSCs) into chondroblasts and osteoblasts (among others) which then continue to differentiate under the (anabolic) conditions. The activity of chondroblasts and osteoblasts includes the build-up and strengthening of cartilage and bone and their ECM. The enhanced structural remodeling requires additional nutrients and fluid incorporation by and throughout the ECM at a rate increased by the anabolic environment. This is an intuitive, natural response to an increased requirement for structural support as would be the case for a weightlifter generally, or for an astronaut residing on a planet or moon more massive than the Earth, thus experiencing stronger gravity.

Catabolic activities that occur within and to the ECM include electrochemical signaling and subsequent release of pro-inflammatory cytokines, transformation of human mesenchymal stem cells (MSCs) into chondroclasts and osteoclasts (among others) which then continue to differentiate under the (catabolic) conditions. The activity of chondroclasts and osteoclasts includes the breakdown of cartilage and bone, respectively, and their ECM which is resorbed into the body. This is an intuitive, natural response to a reduced requirement for structural support, as would be the case for wheelchair-bound patients, or for an astronaut residing in zero-gravity space for prolonged periods of time.

Revisiting the definition of “decompression” from a spinal-remodeling perspective, “decompression” is the mechanical-induction of anabolic growth and reparative processes within areas of pathological interest of the spine via application of amplitude modulation of mechanically-distractive forces acting axially upon the spine which flex and extend the facet and intervertebral joints while continuously unloaded. This activity gives rise to multiple effects. First, it stimulates natural mechanical strain-associated cell-triggers within the ECM which electrochemically signal the generation and release of anti-inflammatory cytokines and growth factors associated with synthesis of new, healthy cartilage, bone and synovial tissues. Second, it effectuates a ‘pumping action’ in the region to enhance fluid and nutrient delivery required for cartilage and bone repair. Third, it acts to withdraw and recover herniated fluids into the spine, allowing damaged annulus tissue to heal. Fourth, it physically unloads pressure on the facet and intervertebral joint spaces, which in turn (a) relieves nerve impingement pain and restoring function, (b) opens joint space and (c) provides a mechanically-unloaded environment within which to heal.

The United States Food and Drug Administration has cleared several therapeutic “spinal decompression” medical devices for safe effective use by patients. 89 ITH is the Product Code assigned to modern (as of this writing) spinal decompression devices, which has the Classification Name: “Power Traction Equipment”. The Indications for Use for one such device reads as follows: “The DRX9000 True Non-Surgical Spinal Decompression System provides a primary treatment modality for the management of pain and disability for patients suffering with incapacitating low back pain and sciatica. It is designed to apply spinal decompressive forces to compressive and degenerative injuries of the spine. It has been found to provide relief of pain and symptoms associated with herniated discs, bulging or protruding intervertebral discs, degenerative disc disease, posterior facet syndrome and sciatica.” (Source: FDA Online Access, FDA Clearance #K060735, Retrieved Oct. 25, 2021: https://www.accessdata.fda.gov/cdrh_docs/pdf6/K060735.pdf)

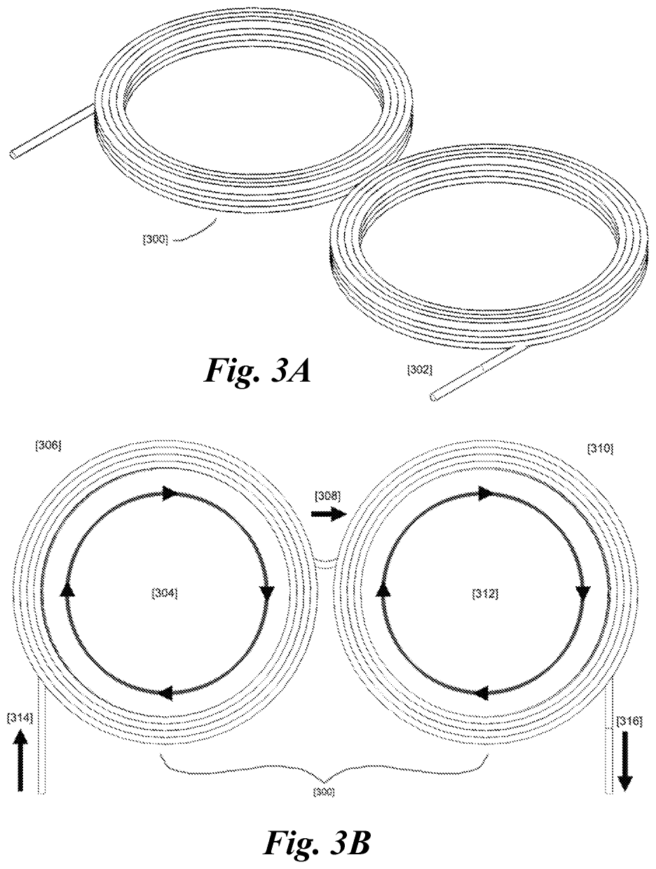

According to the present disclosure, mechanical spinal decompression is combined with a pulsed electromagnetic field (PEMF) in order to enhance the effectiveness of the therapy. FIGS. 3A and 3B depict one embodiment of a “twin-coil” 300 used to produce pulsed electromagnetic fields. Other coil designs which may be used include “butterfly coils”, single-coil or coil(s), and Helmholtzcoils. Conductive wire 302, typically a solid-core copper wire coated in a dielectric, is coiled about a central axis 304 in a uniform manner, as shown in FIG. 3B. In one embodiment, the central diameter of each coil 306/310 is approximately four inches. In another embodiment, the central diameter of the coils is between one and less than four inches. In another embodiment, the diameter of the coils is less than ten inches. When a sufficient number of turns is achieved in the “A-side” coil 306, the wire 302 is passed through 308 to the second coil, the “B-side” coil 310. The B-side coil is turned about the B-side coil axis 312.

Electrical power communicates with the twin-coil 300 as shown by the black arrows 314 and 316 in FIG. 3B. The electrical power is shown flowing through the A-side coil first, orbiting central axis 304 before passing 308 into the B-side coil. Electric power then flows through the B-side coil, orbiting central axis 312.

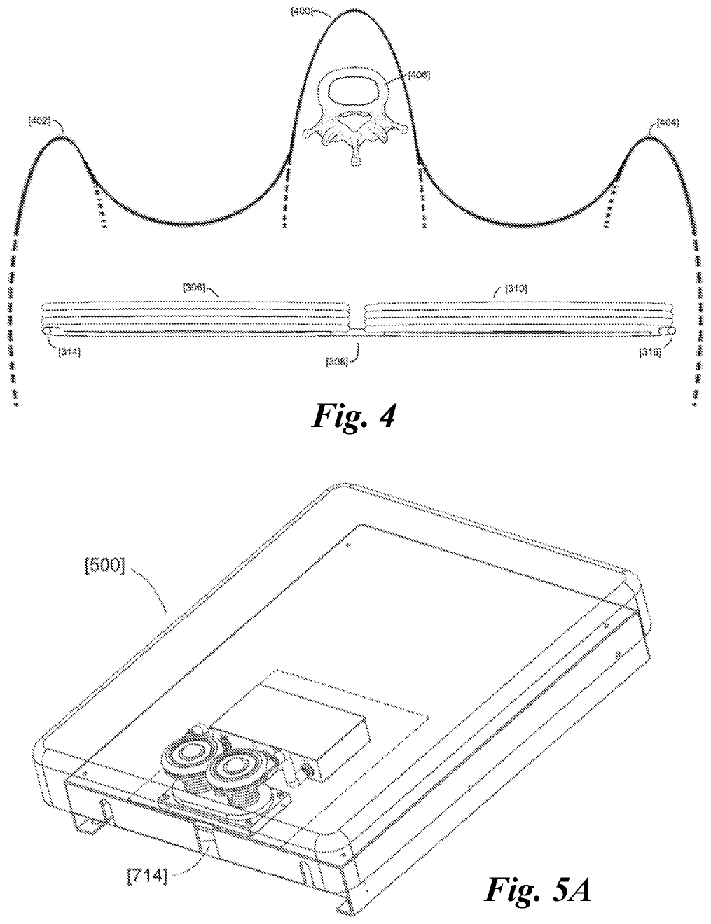

FIG. 4 depicts twin-coil 300 lying flat, wire ends 314 and 316 extending towards the viewer. Upon application of electrical power to ends 314 and 316, as depicted in 320, a magnetic field 400/402/404 is formed. The magnetic field surrounds the coiled wire, having magnetic intensity peaks 402 and 404 corresponding to the outermost region of the coils 306/310. The magnetic field generated by the A-side coil 306 and the magnetic field generated by the B-side coil 310 add in proportion to the distance between their coils, resulting in a peak magnetic intensity 400 about 308.

Placement of the twin-coil within the upper-mattress section 110 and beneath the AoL 108 of the lumbar spine is intended to target the maximum pulsed electromagnetic field (PEMF) intensity 400 upon, within and surrounding the pathological region of interest 406.

The design of the coils suggested herein is given as a range of general characteristics currently found in technology in-use today. The design of the coils will be specific to each application's end-effecting apparatus, and specific desired clinical outcomes.

The use of a pulsed electromagnetic field is believed to generate mechanical strain upon the highly ionically-charged ECM of the skeletal tissue. Such electro-kinetic events may also generate electro-chemical cell signaling, leading to new tissue synthesis and release of anti-inflammatory cytokines. The use of PEMF is also believed to upregulate cartilage and bone synthesis in a variety of joint and bone presentations and locations, including the spine.

The pulsed electromagnetic field (PEMF) therapeutic device of the present disclosure generates a pulsing magnetic field extending invisibly into the body. Ionically-charged structures within tissues exposed to PEMF are physically deflected within the field according to its amplitude and rate-of-pulsation (a.k.a. its ‘frequency’). Within the pulsing magnetic field, this electrokinetic activity is translated into electro-chemical cell signals that trigger physiological modifications.

The term ‘pulsing’ used in reference to the PEMF means to cycle the intensity, or amplitude, of the magnetic field, as used herein unless otherwise described. Frequency modulation is also contemplated within the scope of this disclosure. The frequencies and amplitudes may vary by application. In certain embodiments, the device can create pulsing magnetic fields with one or more frequencies between 0.001 Hz and 100 Hz. In other embodiments, the device emits fields with frequencies up to 300 Hz. In certain embodiments, the device will scan through frequencies within the range of 0.01 Hz and up to 300 Hz. The device produces magnetic fields with intensities ranging from 70 uT to 1000 uT. In other embodiments the device produces field intensities up to 2.5 mT. In other embodiments, the device produces field intensities up to 12 mT.

Decompression therapy using cyclical (a.k.a. ‘amplitude modulated’) mechanical forces in combination with PEMF therapy, using pulsed (a.k.a. ‘amplitude modulated’) magnetic fields, both activate the anabolic signaling events which translate into reparative and restorative spinal remodeling.

Cell membrane adenosine receptors (ARs) are believed to be a target of PEMF excitation in inflammatory cells, but other mechanisms may also play a role. It is believed that there may be an increase in the density of two ARs, A2A and A3, on the cell membranes of human chondrocytes, synoviocytes and osteoblasts following PEMF exposure. In this way, PEMF may cause ‘agonist’ activity for the A2A and A3 AR pathways, acting to upregulate their activity. Upregulated A2A and A3 activity may inhibit the NF-κB pathway, which may in turn inhibit both matrix metalloproteinase synthesis and pro-inflammatory signaling.

Cartilage cells, in the presence of both a specific pharmacological A2A receptor agonist as well as PEMF stimulation, may produce increased anabolic products. A2A stimulation via agonist drugs may have a chondroprotective effect, reducing joint inflammation and cartilage damage in septic arthritis patients. Upregulated A2A and A3 activation may be expressed biomechanically through enhanced new synthesis of proteoglycans and ECM, differentiation of MSCs into chondroblasts and osteoblasts, and synthesis of new cartilage, bone, and synovial tissues and fluid. This activity may be protected through synthesis and release of anti-inflammatory cytokines.

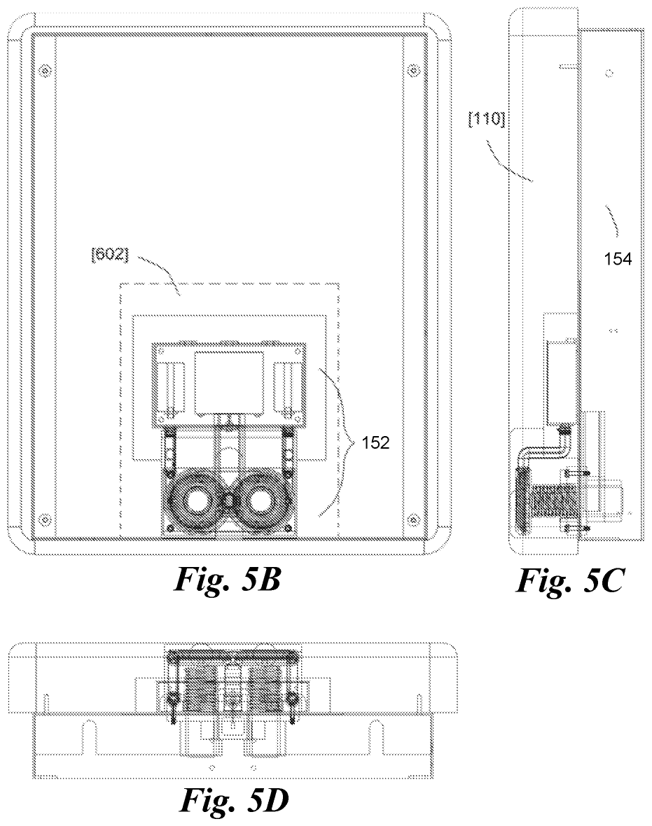

FIGS. 5A, 5B, 5C and 5D are oblique, top, end and right side views, respectively, of one embodiment of PEMF device 152, positioned such that axes 304 and 312 of twin-coil 300 are essentially normal to the surface of the upper-mattress section 110 of the split-mattress 106.

FIG. 5A depicts an isometric view of PEMF device 152 positioned within the upper mattress section 110, in the end normally facing the lower mattress section 112. PEMF device 152 is shown affixed to an upper frame 154. A rectangular section 602 of the upper frame is shown as reference.

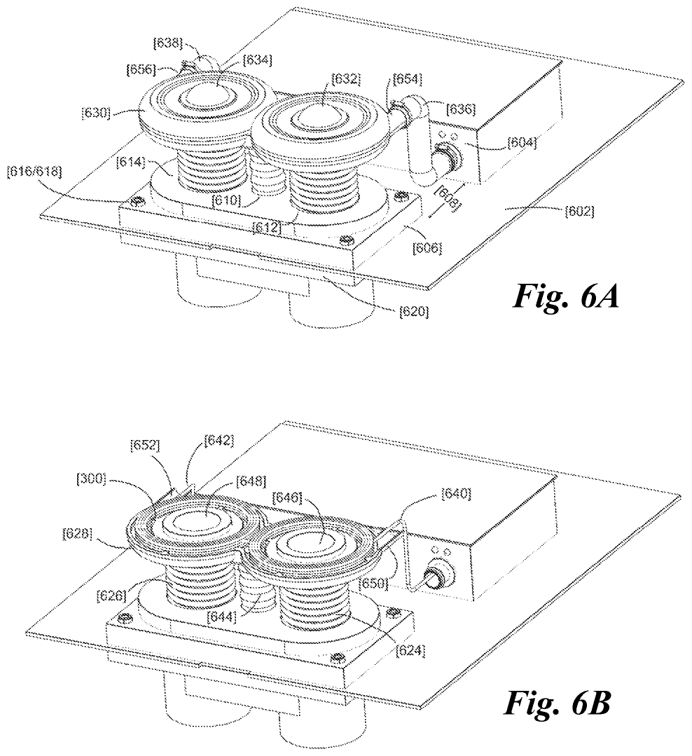

FIGS. 6A and 5B depict front-left oblique views of PEMF device 152 mounted upon a section 602 of the upper frame 154. PEMF module 604 is shown centered-on and affixed to section 602. In one embodiment, PEMF module 604 measures approximately 11W×6L×2.25T inches. PEMF module 604 faces the upper manifold 606 at a distance of approximately 3 inches, referred to herein as the “keep away” distance 608. In other embodiments, the distance 608 is between 1 and 3 inches. In other embodiments, the distance 608 is greater than 3 Inches and up to 12 inches. The “keep away” distance in this example is the distance beyond which concern over metallic and electronic components is reduced. The region of peak magnetic field intensity 400 is substantially-influential within a space which intersects section 602 at the “keep away” distance. PEMF module 604 is placed beyond the “keep away” region in one embodiment of the present disclosure.

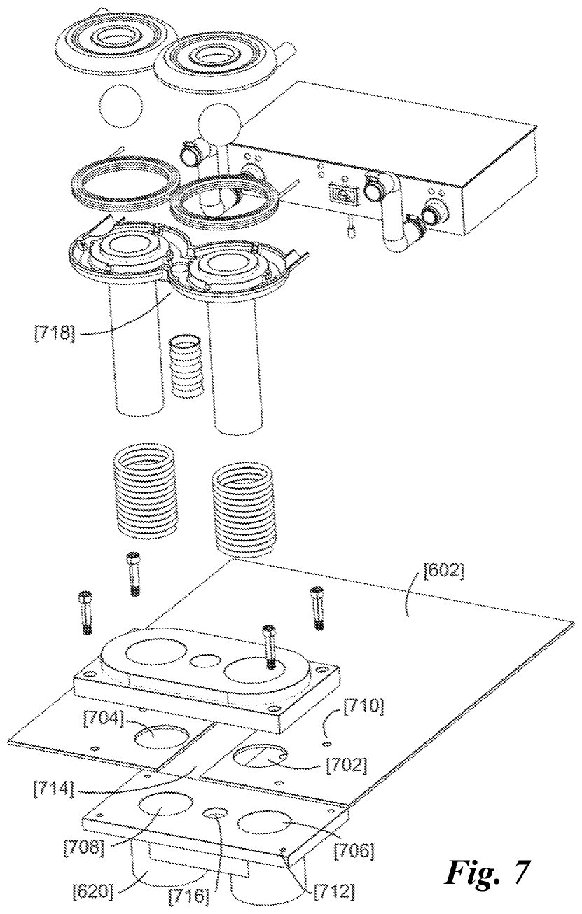

FIG. 7 is an exploded view of one embodiment of the present disclosure. The exploded view is that of view 600. It is useful to reference FIGS. 6A, 6B and 7 for the detailed description below.

The upper manifold 606 is composed of a non-metallic material, such as blue acetal copolymer, and may be machined, molded, 3D printed or otherwise constructed. In one embodiment, the upper manifold 606 is approximately 9.6W×5L×1.3T inches. It contains three holes drilled vertically therethrough all along the widthwise central axis as: a) central hole 610 1.5 inches in diameter, b) two side holes 612 and 614 each 2.5 inches in diameter and set 2.3 inches away from the central hole 610 on either side. Four counterbore screw clearance holes 616 for 5/16-18 socket cap head screws are drilled through at four corner locations set 8.6 by 4 inches apart. Four glass-filled nylon (non-metallic) 5/16-18 socket cap head screws 618 are shown fully-tightened and are of sufficient length to clamp section 602 (via four through holes 710) between the upper manifold 606 and the lower manifold 620 (via four holes 712 having at least partial 5/16-18 threading). A slot 714 is cut into the upper frame 154/section 620 approximately two inches wide and extending approximately to the intersection of the “keep away” region. Any structural integrity loss is accounted for by the clamping of 606 and 620.

Two non-metallic springs 624 and 626 with outer diameters of 2.4 inches, inner diameters of 2.0 inches, and uncompressed lengths of approximately 3.3 inches are shown in view 622. The compressed length of the springs is approximately 1.3 inches. In one preferred embodiment, the springs' ends are closed, squared and ground. The springs' bases are affixed to section 602 and at their other ends to twin coil support 628. In other embodiments the springs 624 and 626 are attached only to either 602 or 628 or are not attached to either. Springs of this type are in common use and are fabricated using plastic materials including polyetherimide (PEI), which are non-metallic and exhibit outstanding mechanical strength and thermal performance. The springs 624/626 in one embodiment have a minimum rate of 3 pounds per inch (lbs./in). In other embodiments, springs 624/626 have minimum rates ranging from 1 and up to 3 lbs./in, and in other embodiments from 3 and up 10 lbs/in.

The design of the compression rate (lbs./in) of the springs is sufficient to support the combined weight of twin-coil support 628, twin coil 300, PEMF cover 630, spacing spheres 632 and 634, A-side cooling hose & clamp 636, B-side cooling hose and clamp 638, A-side electric cable 640, B-side electric cable 642 and exhaust hose 644.

PEMF device 152 as shown in FIG. 6B is the device of FIG. 6A after removal of the spacing spheres 632/634, PEMF cover 630, A- and B-side hoses and clamps 636/638. FIG. 6B shows twin-coil 300 seated within a channel, set into the top of the twin-coil support 628, which allows air to flow below and around the twin-coil for thermal management. Electrical cables 640/642 are shown connected to twin-coil ends 314/316.

Twin coil support 628 is composed of a non-metallic material, such as blue acetal copolymer, and may be machined, molded, 3D printed or otherwise constructed. In one embodiment, the twin coil support is molded as a single piece. It is organized as two vertical cylinders having outer diameters of 1.9″ and spaced at 2.3″ from center, which corresponds to the centers of holes 612/614 in the upper manifold 606, axes 304/312 of twin-coil 300, through-holes 702/704 in section 602, and through-holes 706/708 in the lower manifold 620, respectively.

As will be described, the two vertical cylinders capture springs 624/626 therethrough, and also against the underside of the upper portion of the twin coil support 628. Additionally, the diameter of holes 702/704 is 2.22 inches, which is sufficient to let the vertical towers of the twin-coil support 628 pass through but which stops the springs 624/626.

The springs allow restorative vertical displacements of the twin-coil support in response to the patient's weight from above the upper-mattress section 110. The two vertical cylinders of the twin-coil support move freely through the springs, the section 602 holes 702/704, and the lower manifold holes 706/708. In this manner, the location of the twin-coil is maintained with respect to both the level of the spine receiving PEMF (x and y axes of mattress), as well as to the vertical distance between the twin-coil and the patient's spine.

In FIG. 6B, the outer A- and B-side hoses have been omitted to show their corresponding internal electrical cables 640/642, which are run therethrough in the normal course. The upper portion of the twin-coil support 628 extends to either side of the vertical towers and ends with lower “half-ports” 650/652. A-side electric cable 640 is shown connecting A-side coil 306 to the corresponding A-side port (as shown) on the PEMF-module 604. B-side electric cable 642 is shown connecting to B-side coil 310, and (not shown) to the corresponding B-side port on the PEMF module 604. Two hemispherical dugouts 646/648 are formed centered about the A-side axis 304 and B-side axis 312. Two 1.7-inch diameter spacing spheres 632 and 634 in one embodiment, are placed within appropriately larger diameter hemispherical dugouts where they can freely spin. The spacing spheres are intended to preserve a minimum of physical distance from the top of the twin-coil 300 to the undersurface of the upper mattress section 110. The spheres may be composed of a non-metallic material, such as blue acetal copolymer, and may be machined, molded, 3D printed or otherwise constructed. Benefits of planning for minimum separation from the twin-coil include preserving the structural integrity of the twin-coil itself, ensuring target magnetic field strength intensity 400 throughout treatment, and maintaining an air-flow channel all around the twin-coil 300 for thermal management.

The PEMF cover 630 is a molded part which mates to the top of the twin coil support 628. The inner cavity of the PEMF-cover is a rounded, circular channel similar to that of twin coil support 628 and also contains spacing fins that preserve the positioning of twin-coil 300 and allow cooling air to flow above and around twin-coil 300. The cover is shown having two holes, centered above corresponding spacing spheres 632/634, allowing the spheres to pass through the cover approximately 0.5 inches above the top of the cover, and to spin freely within the hemispherical dugouts 646/648. In other embodiments, the PEMF cover 630 incorporates a molded or machined dome(s) directly into the part, eliminating the spacing spheres and hemispherical dugouts. At either end of the cover two upper half-ports 654/656 are formed which correspond to the lower half-ports 650/652, and over which the cooling hoses and clamps 636/638 are fit and clamped. The ports formed of the upper and lower half-ports are approximately 0.70 inches OD/0.60 inches ID. The cooling hoses are designed to fit appropriately.

In normal use, PEMF device 152 is generating a pulsing magnetic field through conversion of a portion of the electrical power, passed through twin-coil 300, into magnetic field energy. A portion of the electrical power is converted into heat and must be managed to ensure safe operation. Thermal management is accomplished through active air cooling in one embodiment of the present disclosure. Aside and B-side cooling hoses & clamps 636/638, constructed of non-metallic materials, are used to carry pressurized cooling air from the PEMF-module 604 into the space formed between the twin-coil support 628 and PEMF cover 630.

Pressurized air is brought in through both hoses 636/638 and is circulated over, under, and around both sides of twin-coil 300. Heat from twin-coil 300 is pumped out through the central exhaust port 718 and into exhaust hose 644. The exhaust hose 644 in one embodiment is secured via adhesive, clamping or other means to the port in the center of the twin-coil support 628. As shown in views 600/622, the hose extends downward into the central hole 610 of the upper manifold 606 where it can exhaust heated-air down into the lower-manifold 620 through hole 716.

Cooling hoses 636/638 also carry electrical power via electrical cables 640/642 which are run therethrough. In other embodiments, the electrical cables 640/642 are run outside of the cooling hoses. Both the cooling hoses 636/638 and electrical cables 640/642 are flexible and move smoothly throughout the full vertical range-of-motion of the twin-coil support 628 upon springs 624/626.

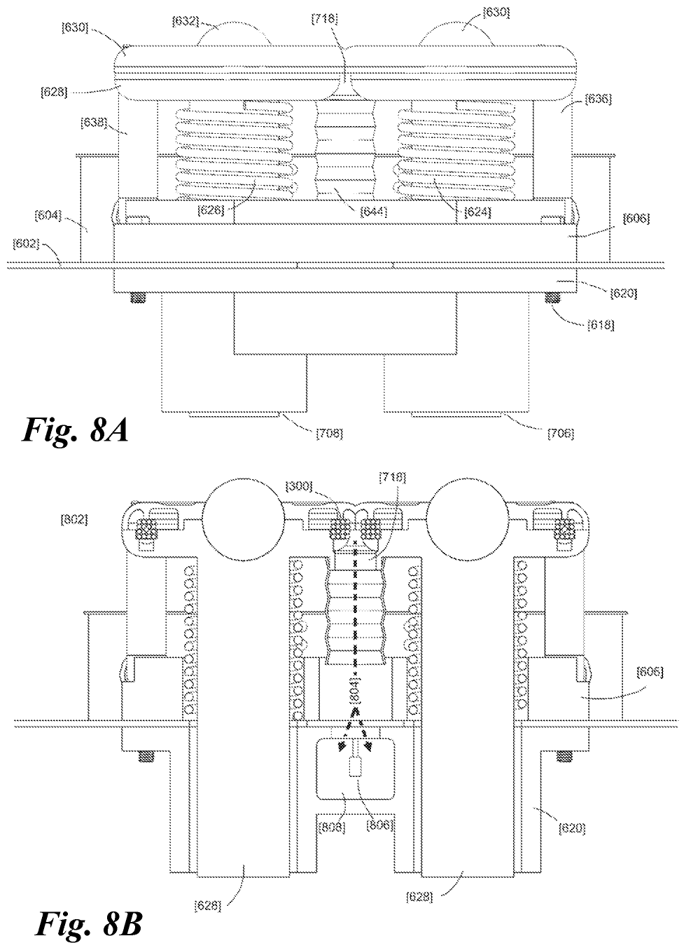

FIGS. 8A and 8B depict front views of one embodiment of the present disclosure. FIG. 8A shows the front view of the front-left views of FIGS. 6A and 6B. FIG. 8B is a cutaway view through the widthwise center plane of the twin-coil support 628. PEMF device 152 is portrayed in the fully-raised position, which is the fully-uncompressed length of the springs 624/626. Air brought in from the PEMF module 604 through the hoses 636/638 circulates through the passageways formed between the PEMF cover 630 and top of the twin-coil support. The circulating air 804 carries heat from twin coil 300 out through the central port 718, through exhaust hose 644, through hole 610, cutaway 714 and into the lower manifold 620. Heated exhaust air 804 is received into exhaust pipe 808 and finally out, below section 602. As exhaust air 804 moves out of 808, it passes by temperature sensor 806 which is monitored by the PEMF module 604. The PEMF module 604 utilizes exhausted air 804 temperature data to regulate aspects of the cooling air supply including flow rate, pressure, temperature, and humidity.

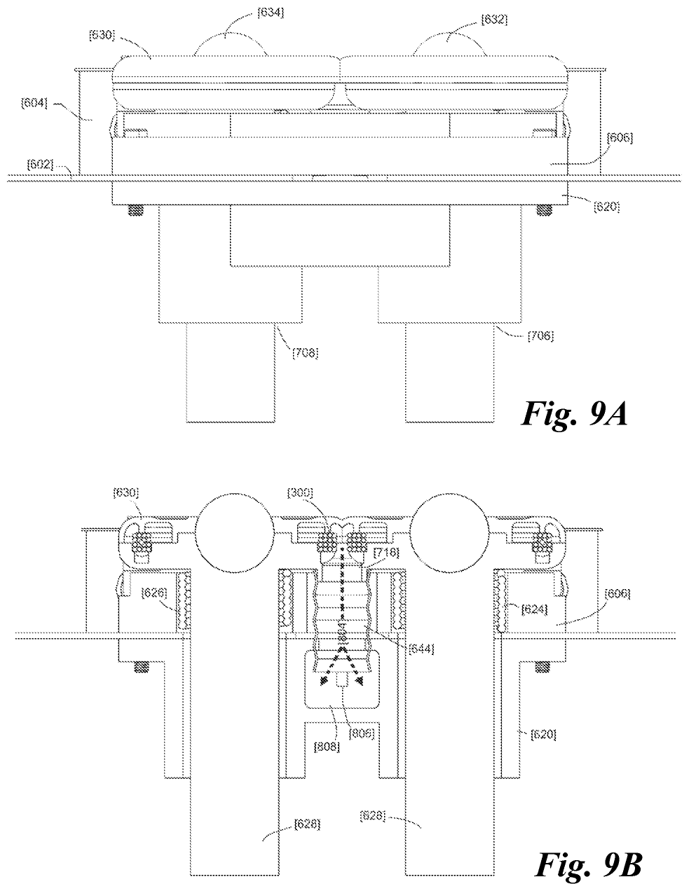

FIGS. 9A and 9B depict front views of one embodiment of the present disclosure. FIG. 9A is the front view of the front-left views of FIGS. 6A and 6B. FIG. 9B is a cutaway view through the widthwise center plane of the twin-coil support 628. PEMF device 152 is portrayed in the fully-lowered position, which is the maximally-compressed length of the springs 624/626. Air brought in from the PEMF module 604 through the hoses 636/638 circulates through the passageways formed between the PEMF cover 630 and the top of the twin coil support. The circulating air 804 carries heat from twin-coil 300 out through the central port 718, through exhaust hose 644, through hole 610, cutaway 714 and into the lower manifold 620. Heated exhaust air 804 is received into exhaust pipe 808 and finally out, below section 602. As exhaust air 804 passes out of exhaust pipe 808, it passes by temperature sensor 806 which is monitored by the PEMF module 604. The PEMF module utilizes exhausted air 804 temperature data to regulate aspects of the cooling air supply including flow rate, pressure, temperature, and humidity.

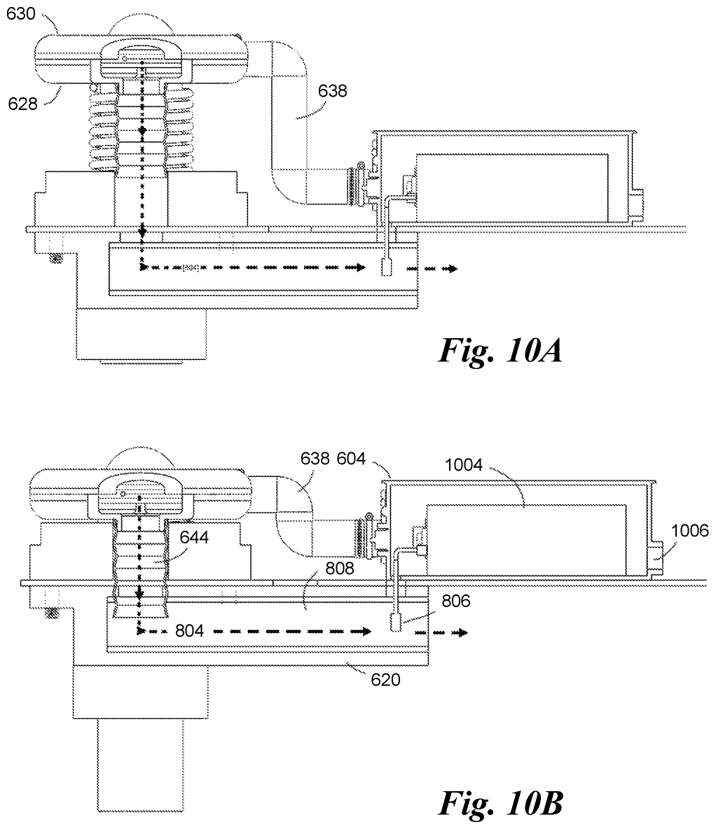

FIGS. 10A and 10B are left-side views of PEMF device 152 cut down the center lengthwise plane of the PEMF module 604. FIG. 10A shows the PEMF-device in the fully-raised position. FIG. 10B shows the PEMF-device in the fully-lowered position. In both cases, cooling air is brought in from the PEMF module through hoses 636/638 and into the space between PEMF-cover 630 and the top of the twin coil support 628. At the midplane between the A-side and B-side coils 306/310 heated air 804 is pushed out through central port 718, through exhaust hose 644 and upper manifold 606 through hole 610, and into the lower manifold 620 exhaust pipe 808. Exhaust hose 644 passes through unimpeded throughout the range-of-motion of the twin-coil support 628, and so needs only be secured to the central port 718. Heated air 804 passes by air-temperature sensor 806 before leaving the lower manifold. The temperature sensor is connected to the PEMF-driver 1004.

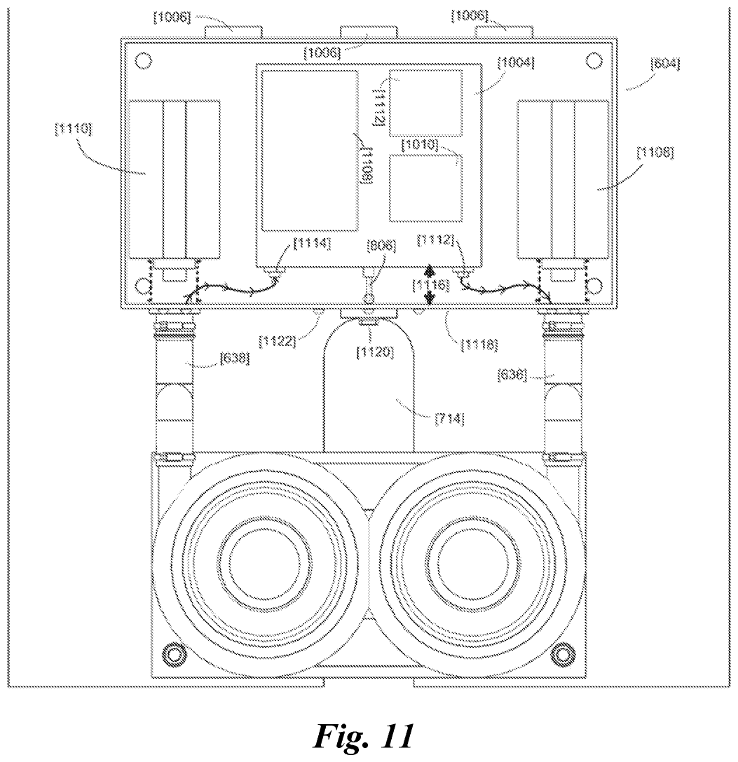

FIG. 11 is a top-down view of PEMF device 152. In this figure, the lid of the PEMF module 604 has been removed showing two air-pumps 1108/1110 and the PEMF driver 1004. The PEMF driver 1004 is an enclosure which contains electronics 1008 which generate the electrical waveforms 1112/1114 which drive twin-coil 300, read the temperature sensor 806, and manipulate injection of cooling air into cooling hoses 636/638. The PEMF-driver 1004 contains a microprocessor 1010 and software 1012 which control all aspects of PEMF device 152. The microprocessor and software control the electronics, which control the pumps and the electric waveform electrical waveforms 1112/1114 generation. The microprocessor communicates 1116 with an LED panel 1118 which displays statuses via indicators 1122 including power, pump states, waveform generators, external communications, and errors. Additionally, the microprocessor 1010 may be in communication with external devices through port 1006, or via wireless protocol. External devices may fully control the PEMF device 152 or may exchange information with PEMF device 152. Panel 1118 contains a user-serviceable fuse 1120 which interrupts power to PEMF device 152.

The air pumps 1108/1110 in one embodiment are used to affect airflow into cooling hoses 636/638. In this embodiment the air pumps intake fresh air from port 1006, above section 602, which is the upper frame 154. The air then circulates across twin-coil 300 exchanging heat which is then exhausted through central port 718, through exhaust hose 644 and out through exhaust pipe 808. The exhausted air 804 is let out underneath section 602, which is the upper frame 154. In this embodiment the intake and exhaust are separated from each other. In other embodiments, cooling air may be delivered by sources including fans, blowers, air compressor(s), and external connection to pressurized gas.

The device disclosed herein is advantageous for a number of reasons. Patients rarely have the opportunity to receive both spinal decompression and EMF treatment modalities, due partially to the economics involved for healthcare providers. Normally, PEMF therapy would need to be scheduled separately from spinal decompression therapy, and this assumes that the same location would have both device types available. Staff familiar with the operational characteristics of both devices would be required to administer the separate treatments. Economically speaking, the return on investment for providing both treatment modalities may not make an attractive proposition for all healthcare facilities. The device of the present disclosure addresses this problem by combining both therapies into a single treatment device. In some embodiments, the user interface of the spinal decompression device may incorporate all of the necessary interaction with and for the control and instruction of the PEMF-device.

Both the spinal decompression and PEMF modalities operate on similar electro-kinetic biological pathways, using different stimuli. Simultaneous exposure to both modalities encourages additional anabolic signaling. It is known that the region-of-effect is receiving an upregulated supply of nutrient-rich fluid while undergoing decompression which augments the amount of material affected by the PEMF stimulation.

The device of the present disclosure incorporates the PEMF device into a mattress, providing the patient with enhanced comfort while receiving therapy. Spinal decompression devices (e.g., supine-orientation type) also keep the patient isolated and not otherwise moving. The apex of lordosis is utilized as an index of reference for patient positioning on the table. In this way, the region of pathological interest is also in a known location relative to the spinal decompression device, and therefore the PEMF device can reliably treat at the same lumbar position at each treatment session.

Incorporating both modalities into a simultaneous treatment may yield less overall time spent by the patient at the healthcare facility, enhanced therapeutic action for the same time spent receiving treatment, higher patient compliance due to ease of obtaining both modalities, higher patient compliance due to increased actual (quantitative) improvement in clinical outcomes as compared to either treatment alone, and higher patient compliance due to increased perceived (qualitative) improvement in clinical outcomes as compared to either treatment alone.

Incorporating PEMF into the decompression device allows the device to be used for either treatment individually or both simultaneously. This allows the healthcare provider or owner more flexibility in scheduling patients. The result is improved patient access to treatment by improving clinic flow and operating costs, reduced healthcare spending, and through faster return on investment for the purchase of the device disclosed herein.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refers to at least one of something selected from the group consisting of A, B, C . . . and N, the text should be interpreted as requiring only one element from the group, not A plus N, or B plus N, etc.

What is claimed is:

1. A pulsed electromagnetic field spinal decompression device comprising:
a generally-horizontal mattress having a first end and a second end;
a generally-vertical decompression tower disposed at the first end of the generally-horizontal mattress;
a tension strap having a first end and second end, the first end being operably connected to the decompression tower;
a pelvic harness operably connected to the second end of the tension strap; and
a pulsed electromagnetic field device disposed in the generally-horizontal mattress.
